(19) United States
(12) Patent Application Publication
(10) Pub. No.: US 2025/0346686 A1
(43) Pub. Date: Nov. 13, 2025

(54) HYALURONIC ACID GRAFTED WITH POLY(N-ISOPROPYLACRYLAMIDE) (HA-G-PNIPAM) USE IN TOPICAL MOLECULAR DELIVERY

(71) Applicant: NABSOLUTE CO., LTD., Bangkok (TH)

(72) Inventors: Jittima LUCKANAGUL, Bangkok (TH); Visarut BURANASUDJA, Bangkok (TH); Vikram AHUJA, Bangkok (TH); Kittima LEKMANEE, Bangkok (TH); Kittipong SANOOKPAN, Ubon Ratchathani (TH); Peerawas KOPONGPANICH, Bangkok (TH)

(21) Appl. No.: 19/200,811

(22) Filed: May 7, 2025

Related U.S. Application Data
(60) Provisional application No. 63/644,075, filed on May 8, 2024.

(51) Int. Cl. C08B 37/08; A61K 8/04; A61K 8/91; C08F 251/00; C08J 3/075

(52) U.S. Cl. CPC C08B 37/0072; A61K 8/042; A61K 8/91; C08F 251/00; C08J 3/075; C08J 2305/00

(57) ABSTRACT

This invention outlines the method for preparing hyaluronic acid grafted poly(N-isopropylacrylamide) (HA-G-PNIPAM) nanogel, which involves several key steps. Begin by dissolving 0.10 grams of hyaluronic acid in 100 ml of distilled water while stirring gently. Subject the solution to sonication for 15 to 30 minutes to ensure uniformity. After sonication, transfer the solution to a sealed container and refrigerate it at 4° C. for at least 12 hours to stabilize the mixture. Proper storage of hyaluronic acid at −20° C. before use is essential for maintaining its integrity. Following these steps will yield a quality nanogel suitable for external applications.

### CROSS-REFERENCE TO RELATED APPLICATION

The present application claims the benefit of U.S. provisional patent application No. 63/644,075. The disclosures of the above-mentioned application are incorporated herein by reference in their entirety.

### FIELD OF THE INVENTION

The present invention relates to a method for preparing an external application of hyaluronic acid grafted poly(N-isopropylacrylamide) (HA-G-PNIPAM) nanogel.

### BACKGROUND OF THE INVENTION

This invention involves poly(N-isopropylacrylamide) grafted onto hyaluronic acid, which distinguishes it from similar products due to its specific application for external use.

Many active ingredients in current external products encounter various challenges. To tackle these issues, the inventor has investigated the effectiveness of poly(N-isopropylacrylamide) grafted onto hyaluronic acid, emphasizing its efficiency and the synergistic effects it has with other active ingredients that greatly enhance external product applications.

Although there are other inventions that offer comparable benefits, this particular one has several advantages: it is easy to produce, convenient to use, non-irritating, and safe for the skin.

This innovation showcases the safety and applicability of poly(N-isopropylacrylamide) grafted onto hyaluronic acid, marking a significant advancement for external use products. Its user-friendly, water-based formulation is gentle on the skin, making it an excellent choice for consumers.

### SUMMARY OF THE INVENTION

The present disclosure outlines a comprehensive method for preparing an external application of hyaluronic acid grafted poly(N-isopropylacrylamide) (HA-G-PNIPAM) nanogel. This method consists of several detailed steps to ensure the quality and efficacy of the final product:
1. Dissolution of Hyaluronic Acid: Begin by accurately weighing 0.10 grams of hyaluronic acid (also referred to as Hy-N). Carefully transfer the weighed hyaluronic acid into a clean container. Gradually add 100 ml of distilled or deionized water to the container to facilitate dissolution. It's essential to stir the mixture gently to aid the dissolution process and ensure that the hyaluronic acid is fully integrated into the aqueous phase without any clumping.
2. Sonication Process: Once the hyaluronic acid has completely dissolved, subject the solution to sonication. This step should last approximately 15 to 30 minutes, depending on the desired consistency and uniformity of the solution. The sonication process helps to break down any residual aggregates of hyaluronic acid and promotes homogeneous dispersion in the water, which is crucial for the subsequent formation of the nanogel.
3. Post-Preparation Storage: After completing the sonication step, the solution should be carefully transferred to a suitable storage container with a secure lid. Immediately place the container in a refrigerator set at a temperature of 4° C. for a minimum of one night. This overnight storage is critical for stabilizing the solution, allowing any remaining undispersed particles to settle and enhancing the overall stability of the nanogel formulation.

It is imperative that the solution remains at a constant temperature of 4° C. throughout the entire stabilization period following the overnight storage. Fluctuations in temperature could adversely affect the properties of the hyaluronic acid solution.

Prior to the dissolution process, ensure that the hyaluronic acid solid (Hy-N) is stored properly at a temperature of −20° C. This temperature is necessary to maintain the integrity of the compound during dry storage, preventing degradation or loss of efficacy before it is used in the preparation process.

The overnight stabilization period should be strictly observed, lasting at least 12 hours from the moment the solution is placed in the refrigerator. This ensures sufficient time for the solution to stabilize effectively, preparing it for the next steps in creating the HA-G-PNIPAM nanogel.

Following these detailed steps and specifications will help achieve a high-quality HA-G-PNIPAM nanogel suitable for external applications.

### BRIEF DESCRIPTION OF THE DRAWINGS

Illustrative embodiments of the present disclosure will be described with reference to the accompanying drawings.

FIG. 1 shows the Cytotoxicity test results of poly(N-isopropylacrylamide) grafted onto hyaluronic acid on corneal epithelial cells at various concentrations (*p<0.05).

FIG. 2 shows the Cytotoxicity test results of poly(N-isopropylacrylamide) grafted onto hyaluronic acid on human skin cells at various concentrations. (a) Results on human epidermal keratinocytes; (b) Results on human dermal fibroblasts (*p<0.05).

FIG. 3 shows the certification confirming that 2.0% w/v poly(N-isopropylacrylamide) grafted onto hyaluronic acid does not cause skin irritation.

FIG. 4 shows the certification confirming that 2.0% w/v poly(N-isopropylacrylamide) grafted onto hyaluronic acid does not cause allergic reactions.

FIG. 5 shows the melanin inhibition effect of poly(N-isopropylacrylamide) grafted onto hyaluronic acid in melanocytes stimulated with α-MSH.

FIG. 6 shows Effects of poly(N-isopropylacrylamide) grafted onto hyaluronic acid on wound healing in human epidermal cells (***p<0.001).

FIG. 7 shows the anti-inflammatory effect of poly(N-isopropylacrylamide) grafted onto hyaluronic acid on human epidermal cells (#p<0.05 compared to the control group; * p<0.05 compared to the UV-A-stimulated control group).

FIG. 8 shows the inhibition of collagen degradation by poly(N-isopropylacrylamide) grafted onto hyaluronic acid in rat fibroblast cells.

FIG. 9 shows the antioxidant effect of poly(N-isopropylacrylamide) grafted onto hyaluronic acid in human epidermal cells (** p<0.01 compared to the control group).

FIG. 10 shows the survival of human epidermal cells after exposure to UV-A radiation, comparing cells treated and untreated with poly(N-isopropylacrylamide) grafted onto hyaluronic acid (*** p<0.001 compared to the control group).

FIG. 11 shows the skin penetration study result using a 3D skin model (Episkin™) in combination with Fluorescein Isothiocyanate (FITC).

FIG. 12 shows the anti-inflammatory effect of poly(N-isopropylacrylamide) grafted onto hyaluronic acid loaded with Multi-ex when exposed to UV-A radiation in human epidermal cells.

FIG. 13 The effect of poly(N-isopropylacrylamide) grafted onto hyaluronic acid, which reduces inflammation by 85% when used in combination with standard extracts of Centella asiatica, onion, and licorice (Hy-N-CAG), on nitric oxide production in LPS-stimulated RAW 264.7 cells.

FIG. 14 shows the anti-inflammatory effect of poly(N-isopropylacrylamide) grafted onto hyaluronic acid loaded with standard extracts of Centella asiatica, onion, and licorice (Hy-N-CAG) through the inhibition of nitric oxide synthase in white blood cells.

FIG. 15 shows the anti-inflammatory effect of poly(N-isopropylacrylamide) grafted onto hyaluronic acid loaded with standard extracts of Centella asiatica, onion, and licorice (Hy-N-CAG) through the inhibition of cyclooxygenase in white blood cells.

FIG. 16 shows the inhibition of collagen degradation by poly(N-isopropylacrylamide) grafted onto hyaluronic acid loaded with Multi-ex when stimulated with UV-A radiation. (a) Human epidermal cells; (b) Human fibroblasts.

FIG. 17 shows the effectiveness of poly(N-isopropylacrylamide) grafted onto hyaluronic acid loaded with cannabinoids in inhibiting free radicals in human epidermal cells (*p<0.05, *** p<0.01 when compared to cannabinoids and poly(N-isopropylacrylamide) grafted onto hyaluronic acid loaded with cannabinoids).

FIG. 18 shows the effect of poly(N-isopropylacrylamide) grafted onto hyaluronic acid loaded with standard extracts of Centella asiatica, onion, and licorice (Hy-N-CAG) on oxidative stress induced by H₂O₂ in keratinocyte skin cells (p<0.001).

FIG. 19 shows the effect of poly(N-isopropylacrylamide) grafted onto hyaluronic acid loaded with standard extracts of Centella asiatica, onion, and licorice (Hy-N-CAG) in reducing DCF levels in keratinocyte skin cells induced by H2O2, with the mean±standard deviation (S.D.) (p<0.05).

FIG. 20 shows the UV-A protective efficacy of ascorbic acid encapsulated in poly(N-isopropylacrylamide) grafted onto hyaluronic acid in human epidermal cells.

FIG. 21 shows the UV-A protective efficacy of niacinamide encapsulated in poly(N-isopropylacrylamide) grafted onto hyaluronic acid loaded with niacinamide in human epidermal cells.

FIG. 22 shows the level of MMP-1 expression in keratinocyte cells when stimulated with UV-A radiation, comparing conditions with and without poly(N-isopropylacrylamide) grafted onto hyaluronic acid loaded with standard extracts of Centella asiatica, onion, and licorice (Hy-N-CAG).

FIG. 23 shows the effect of poly(N-isopropylacrylamide) grafted onto hyaluronic acid loaded with standard extracts of Centella Asiatica, onion, and licorice (Hy-N-CAG) on wound healing in human epidermal cells.

FIG. 24 shows morphological characteristics of filopodia on keratinocyte cells.

FIG. 25 shows the efficacy of poly(N-isopropylacrylamide) grafted onto hyaluronic acid loaded with fibroblast growth factor (FGF) in enhancing the skin elasticity of volunteers.

FIG. 26 shows the efficacy of poly(N-isopropylacrylamide) grafted onto hyaluronic acid loaded with stabilized vitamin C (AA2G) in enhancing skin brightness in volunteers.

FIG. 27 shows the skin moisture rate of volunteer groups after applying a gel of poly(N-isopropylacrylamide) grafted onto hyaluronic acid loaded with standard extracts of Centella asiatica, onion, and licorice (Hy-N-CAG), compared to a placebo gel.

FIG. 28 shows the level of irritation at different time points when stimulated with tape, before and after applying the sample.

FIG. 29 shows the percentages of skin moisture in volunteers during the application of the sample on days 6, 14, and 28.

FIG. 30 shows the bar graph illustrating the skin elasticity before and after 28 days of applying the sample, comparing poly(N-isopropylacrylamide) grafted onto hyaluronic acid loaded with standard extracts of Centella asiatica, onion, and licorice (Hy-N-CAG) gel to the placebo gel.

FIG. 31 shows the level of skin roughness before and after applying the sample.

FIG. 32 shows the level of scar roughness in volunteers, comparing before and after applying the gel sample.

FIG. 33 shows the darkness of scar tissue on the skin before and after applying the sample.

FIG. 34 shows the skin moisture level after 30 minutes of applying the sample to the skin.

FIG. 35 shows the level of irritation at different time points when stimulated with tape stripping, before and after applying the sample.

FIG. 36 shows the percentage of skin moisture in volunteers during the application of the sample on days 14 and 28.

FIG. 37 shows the percentage of skin brightness improvement in volunteers during the application of the sample on days 14 and 28.

FIG. 38 shows Skin elasticity before and after 28 days of applying the sample, comparing gel of 5% poly(N-isopropylacrylamide) grafted onto hyaluronic acid nanogel and gel of 0.1% hyaluronic acid.

FIG. 39 shows the photo of volunteer number 05.

FIG. 40 shows the result of irritation relief from the product and the untreated area on volunteer number 07.

FIG. 41 shows the skin condition before and after treatment with the product, as well as the untreated area on volunteer number 07.

### DETAILED DESCRIPTION OF THE INVENTION

In the following, preferred embodiments of the present invention will be described in detail with reference to the accompanying, exemplary diagrams.

The process for preparing Hy-N Sterile involves three main steps: (i) Dissolution, (ii) Sonication, and (iii) Post-Preparation Storage. It is essential to follow the protocol precisely; any missing or altered steps may result in an ineffective solution, as indicated by the findings disclosed in this application.

The inventor specifies that Hy-N solid must be stored at a temperature of −20° C. in a controlled, dry environment. This is essential for maintaining the compound's stability, integrity, and overall effectiveness for future use. It's important to keep the material in a protective container that is resistant to moisture and light to enhance its longevity. Additionally, regularly monitor the temperature of the storage facility to ensure these conditions are met. The preparation procedure will be outlined in the steps below.

### 1. Dissolution of Hy-N.

Begin the preparation by carefully weighing out exactly 0.10 grams of Hy-N using a calibrated analytical balance. Accurate measurement is critical and must be performed in a dedicated area to minimize the risk of cross-contamination.

Under a sterile hood, which should be certified for sterility and well-maintained to reduce the risk of airborne contaminants, carefully add the measured Hy-N powder into a sterile container holding 100 mL of sterile ultrapure water. It is essential that both the water and the container are free from any contaminants, as impurities can compromise the solution's quality.

Once the solid has been added to the water, securely close the container, ensuring it is airtight to maintain sterility. After sealing, cautiously remove it from the sterile hood, being mindful to avoid any contact with non-sterile surfaces.

### 2. Sonication.

Proceed to sonication by utilizing a properly calibrated and thoroughly sanitized ultrasonic cleaner or sonicator for this step. Ensure that the sonicator has been tested to confirm it is functioning correctly as this will prevent the introduction of contaminants.

Subject the Hy-N solution to sonication for approximately 15 to 30 minutes, during which you must continuously monitor the solution. Look for visual signs that indicate complete dissolution, such as clarity and the absence of undissolved particles. The objective is to achieve a completely homogeneous and clear solution without any cloudiness or residues, which could impact the efficacy of the final product.

### 3. Post-Preparation Storage:

After sonication is complete, carefully transfer the prepared Hy-N solution into a suitable sterile storage container. Use tools such as sterile pipettes or transfer needles to avoid contamination during this process. Ensure that the storage container is tightly sealed to prevent exposure to the environment, which could lead to contamination or degradation.

Store the resulting Hy-N solution at a temperature of 4° C., which is recommended to preserve its stability. It is crucial to allow the solution to rest and stabilize for a minimum of 12 hours, or overnight if possible, before utilizing it in any experiments or applications. This resting period allows the solute to equilibrate within the solvent, promoting optimal stability and performance in subsequent uses.

When working with this method as described in the invention must keep several important considerations in mind.

First and foremost, all laboratory instruments and equipment must be thoroughly sterilized before each use. This includes not only the containers but also any tools that will come into contact with the solution, such as tubes, bottles, pipettes, and other accessories used during preparation. Strict adherence to sterilization protocols is essential to maintain a contamination-free environment, ensuring the reliability of experimental results.

Additionally, the addition of preservatives, stabilizers, or any other extraneous substances to the Hy-N sterile solution is strictly prohibited. Such substances can compromise the solution's integrity, efficacy, and reliability, potentially resulting in misleading or erroneous experimental outcomes. Always use the solution exactly as prepared to ensure it meets the high standards for purity and effectiveness required for reliable scientific work.

### EMlastic Loading in Hy-N Sterile for In Vitro Testing

### Preparation:

1. Prepare Hy-N sterile by dissolving 0.10 g of Hy-N(solid) in water, as mentioned in the previous protocol.
2. Calculate the stock concentration of EMlastic you want to use.
3. Prepare the EMlastic stock in sterile ultrapure water.

### Example Calculation for 5% Hy-N Sterile:

To prepare a 5% Hy-N sterile solution in a complete medium from 100% Hy-N sterile, use the following equation:

$$C 1 V 1 = C 2 V 2$$

Where:
C1 is 100% Hy-N sterile
V1 is the volume of Hy-N sterile
C2 is 5% Hy-N sterile final concentration to be prepared in complete medium
V2 is the volume of complete medium to be prepared

### Example Calculation for EMlastic:

To prepare 1000 μg/mL of EMlastic from a stock concentration of 500,000 μg/mL, use the same equation:

$$C 1 V 1 = C 2 V 2$$

### Procedure for EMlastic Loading in Hy-N Sterile:

Prepare EMlastic loading in a complete medium in a manner similar to preparing the 5% Hy-N sterile solution. After preparation, mix the EMlastic with Hy-N sterile using a vortex mixer for approximately 3-5 minutes before cell treatment.

### Example Preparation:

To create a medium containing 1,000 μg/mL EMlastic and 5% Hy-N sterile in 2 mL of complete medium, follow these steps:
1. Prepare a stock concentration of EMlastic at 500,000 μg/mL.
2. Prepare the 100% Hy-N sterile as described in the previous protocol.
3. Mix the following in a suitable container:
   4 μL of EMlastic (from step 1)
   100 μL of Hy-N sterile (from step 2)
   1,896 μL of complete medium
4. Vortex the mixture for about 3-5 minutes, and then it is ready for cell treatment.

Note: For in vitro testing, use the solution immediately after preparation.

### Hy-N Safety Test Results

### 1. Conjunctival Cell Toxicity Test

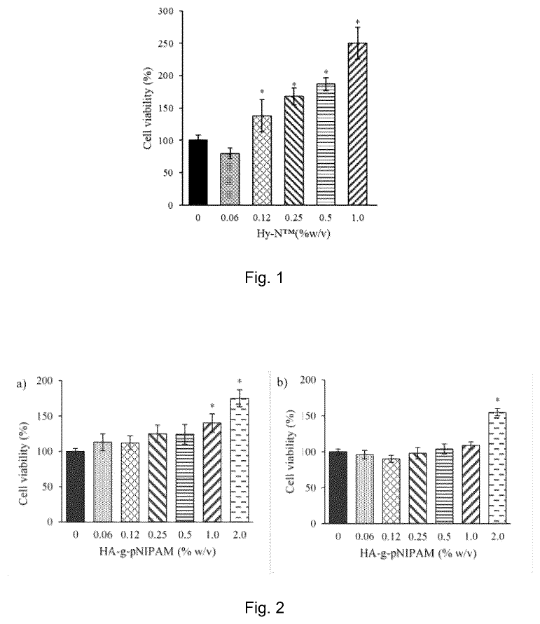

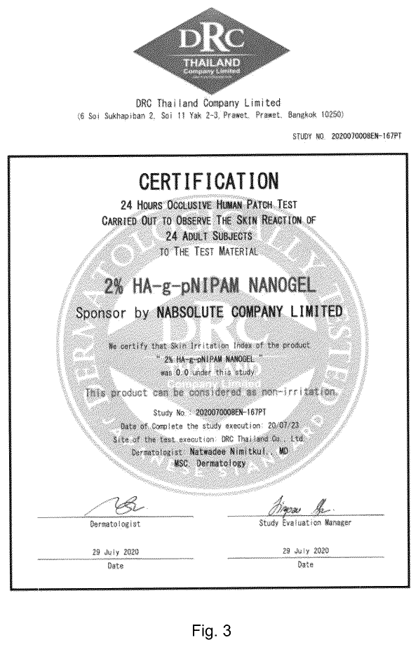

To evaluate the safety of poly(N-isopropylacrylamide) grafted onto hyaluronic acid for human ocular tissue, the inventors' group performed an MTT assay on conjunctival cells. The results showed that cells treated with varying concentrations of poly(N-isopropylacrylamide) grafted onto hyaluronic acid (0.06%-1.0% w/v) for 24 hours remained viable at over 50%. At a concentration of 0.06% w/v, there were no significant changes observed, while concentrations of 0.12% w/v and above showed significant increases in cell viability compared to the untreated control group (FIG. 1). These results clearly indicate that poly(N-isopropylacrylamide) grafted onto hyaluronic acid is a non-irritating product for the human eye.

### 2. Skin Cell Toxicity Test

The safety of poly(N-isopropylacrylamide) grafted onto hyaluronic acid was evaluated using human epidermal stem cells (HaCaT) and human fibroblast (BJ) cells via MTT assay. Cells were incubated with various concentrations of poly(N-isopropylacrylamide) grafted onto hyaluronic acid (0.06%-2.0% w/v) for 24 hours. The results showed that the highest concentration of 0.5% w/v in HaCaT cells (FIG. 2a) and 1.0% w/v in BJ cells (FIG. 2b) did not significantly affect cell survival compared to control groups that did not receive the treatment. While higher concentrations of poly(N-isopropylacrylamide) grafted onto hyaluronic acid increased cell survival, these results indicate that it is a non-irritating product for human skin cells.

### 3. Clinical Skin Irritation Test

This test was conducted by an independent laboratory, DRC (Thailand) Co., Ltd. The irritation study of poly(N-isopropylacrylamide) grafted onto 2.0% w/v hyaluronic acid utilized a 24-hour human skin patch test. After obtaining full consent and confirming the inclusion/exclusion criteria, the expert observed and photographed the test area on the upper back (lateral spine) of the volunteer. The test sample was applied to the volunteer using a patch. After 24 hours, the volunteer removed the patch on their own. A technician photographed the test area at 1 hour (24 hours total) and at 24 hours (48 hours total). After the patch was removed, a dermatologist evaluated the results, including skin reaction assessments, and assigned an irritation score. The study revealed that all 24 adult volunteers had no reaction to the samples (Tables 1 and 2), resulting in a skin irritation score of 0 according to the Sugai (1995) classification. Thus, these clinical test results confirm that poly(N-isopropylacrylamide) grafted onto 2.0% w/v hyaluronic acid does not cause skin irritation.

TABLE 1
The skin response to 2.0% w/v poly(N-isopropylacrylamide)
grafted onto hyaluronic acid after 24 hours.

| | | | White | | Sterile water | | Physiological | |
|---|---|---|---|---|---|---|---|---|
| | 2.0% Hy-N ™ | | Petrolatum | | for Irrigation | | saline solution | |
| Skin Reaction | 24 h | 48 h | 24 h | 48 h | 24 h | 48 h | 24 h | 48 h |
| No reaction | 24 | 24 | 24 | 24 | 24 | 24 | 24 | 24 |
| Slight erythema | — | — | — | — | — | — | — | — |
| Definite erythema | — | — | — | — | — | — | — | — |
| Erythema + edema, papule | — | — | — | — | — | — | — | — |

| | | | | |
|---|---|---|---|---|
| Skin Irritation Score | 0.0 | 0.0 | 0.0 | 0.0 |

TABLE 2
The skin response to 2.0% w/v poly(N-isopropylacrylamide) grafted
onto hyaluronic acid, evaluated using the Human Repeated Insult
Patch Test (HRIPT) to assess allergenic potential.

| | Induction Phase | | | | | | | | | Challenge Phase | |
|---|---|---|---|---|---|---|---|---|---|---|---|
| Skin Reaction | 1 h | 2 h | 3 h | 4 h | 5 h | 6 h | 7 h | 8 h | 9 h | 24 h | 48 h |
| No reaction | 56 | 55 | 56 | 56 | 56 | 56 | 56 | 56 | 56 | 54 | 56 |
| Slight erythema | — | 1 | — | — | — | — | — | — | — | 2 | — |
| Definite erythema | — | — | — | — | — | — | — | — | — | — | — |
| Erythema edema, papule | — | — | — | — | — | — | — | — | — | — | — |
| Total Subject | 56 | 56 | 56 | 56 | 56 | 56 | 56 | 56 | 56 | 56 | 56 |

### 4. Clinical Contact Allergy Test Results

This test was conducted by the independent laboratory DRC (Thailand) Co. Ltd. The contact allergy study of poly(N-isopropylacrylamide) grafted onto 2.0% w/v hyaluronic acid was performed using the Human Repeated Insult Patch Test (HRIPT), which consisted of four main phases: (1) Screening Phase, (2) Induction Phase, (3) Recovery Phase, and (4) Challenge Phase.

Screening Phase: After obtaining consent and confirming the inclusion and exclusion criteria, an expert observed and photographed the test site on the mid-back (lateral spine) of the selected volunteer.

Induction Phase: The volunteer had a test sample applied using a test patch containing an appropriate amount of the sample. After 24 hours, the volunteer removed the patch themselves. The expert then assessed and photographed any allergic skin reactions before applying the next test patch 24 or 48 hours later. This same sample was reapplied by the expert to the same location on the volunteer's back. This procedure was repeated three times weekly for three consecutive weeks (a total of nine sets of application and removal).

Recovery Period: Following the induction phase, all subjects underwent a recovery period lasting 10 to 14 days.

Challenge Phase: Finally, the same test samples were applied to normal skin near the original attachment site from the induction phase, using test patches. Each patch was removed from the volunteer 24 hours after application. Photographs were taken of each volunteer's test site, and a dermatologist assessed the skin for allergic reactions both 1 hour and 24 hours after the removal of the test patch.

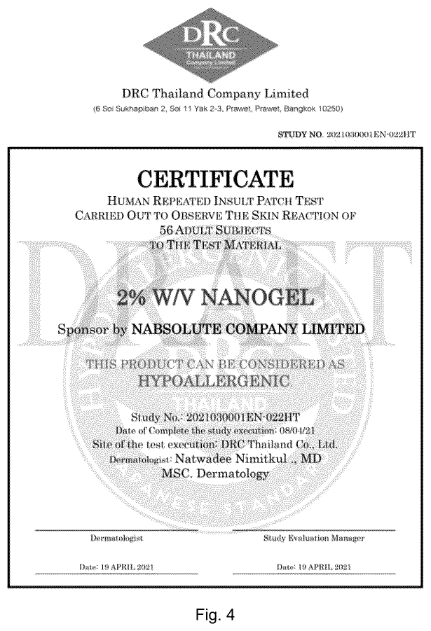

The test results from all 56 volunteers indicated that almost all subjects were free from allergic reactions, with only 1-2 subjects showing mild erythema at the test site, as detailed in Table 2. These results suggest that poly(N-isopropylacrylamide) grafted onto 2.0% w/v hyaluronic acid did not cause contact allergies and could be considered non-sensitizing under the test conditions (refer to FIG. 4).

### Properties of Hy-N as a Modifier of Cosmetic Ingredients

### 1. Whitening Effect

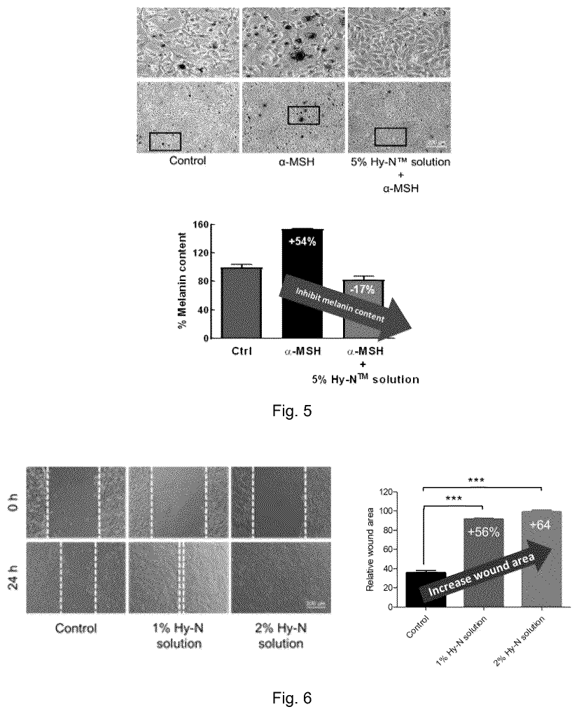

To investigate the whitening effect of poly(N-isopropylacrylamide) grafted onto hyaluronic acid, melanin cells (B16-F10) were used as a model for melanin production. The cells were stimulated with alpha-melanocyte stimulating hormone (a-MSH) at a concentration of 250 nmol and treated with a 5% w/v solution of poly(N-isopropylacrylamide) grafted onto hyaluronic acid for 72 hours. After incubation, the cells were collected and treated with 1 N NaOH solution containing 10% DMSO at 80° C. for 1 hour. The optical density was measured at 405 nm. Results indicated that the poly(N-isopropylacrylamide) grafted on 5% hyaluronic acid significantly inhibited melanin formation by 71% compared to the α-MSH-treated group (FIG. 5).

### 2. Wound Healing Effect

To evaluate the wound healing effect of poly(N-isopropylacrylamide) grafted on hyaluronic acid, an in vitro scratch assay was conducted using human epithelial cells (HaCaT). After overnight culture, the cells were scratched in a straight line and then treated with the poly(N-isopropylacrylamide) solution for 24 hours. The size of the wound was measured after incubation. Findings revealed that solutions of poly(N-isopropylacrylamide) grafted onto hyaluronic acid at concentrations of 1% and 2% w/v enhanced the wound healing efficiency of human epithelial cells by approximately 56% and 64%, respectively (FIG. 6). This study demonstrates that poly(N-isopropylacrylamide) grafted onto hyaluronic acid has potential to promote the wound healing performance of dermal cells.

### 3. Anti-Inflammatory Effect

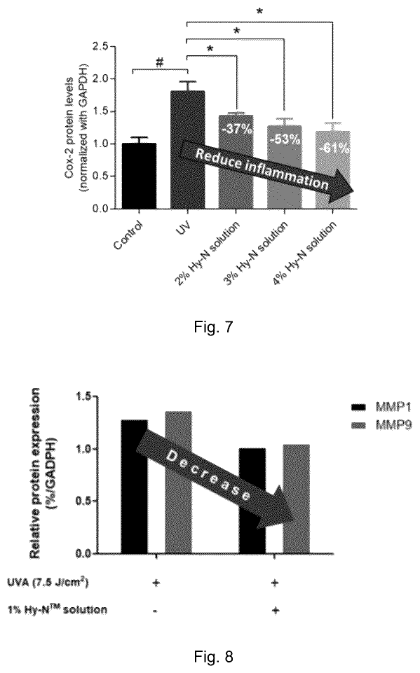

An anti-inflammatory study was conducted using poly(N-isopropylacrylamide) grafted onto hyaluronic acid in human epidermal cells (HaCaT). The cells were cultured with the grafted solution for 24 hours and then exposed to ultraviolet A (UVA) as an inflammatory stimulus. The expression of COX-2 protein, an inflammation marker, was analyzed. The results indicated that poly(N-isopropylacrylamide) solutions at concentrations of 2%, 3%, and 4% significantly reduced COX-2 expression by approximately 37%, 53%, and 61%, respectively, compared to the UVA-irradiated groups (FIG. 7).

### 4. Inhibition of Collagen Degradation

The study examined the anti-wrinkle effects of poly(N-isopropylacrylamide) grafted onto hyaluronic acid using rat fibroblasts as a skin cell model. Ultraviolet A (UVA) was utilized as a stimulator. The expression levels of matrix metalloproteinases-1(MMP-1) and -9 (MMP-9), which are crucial enzymes involved in collagen degradation, were assessed using protein tracking techniques. The findings confirmed that treatment with poly(N-isopropylacrylamide) grafted onto hyaluronic acid decreased MMP-1 and MMP-9 levels after UVA exposure (FIG. 8). These results suggest that poly(N-isopropylacrylamide) grafted onto hyaluronic acid possesses anti-aging properties for dermal cells.

### 5. Antioxidant Properties

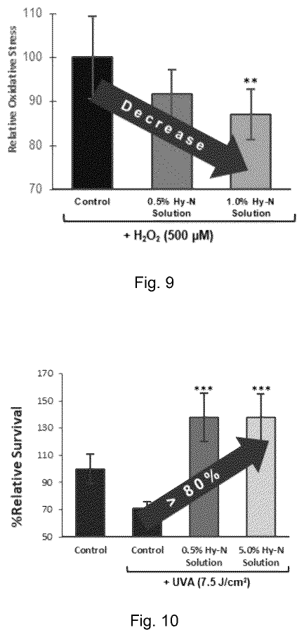

The antioxidant capacity of poly(N-isopropylacrylamide) grafted onto hyaluronic acid was evaluated using the DCFH-DA test. Human epithelial cells (HaCaT keratinocytes) were treated with the grafted solution for 24 hours and then exposed to 500 μM free radicals to induce oxidative stress. The results showed that treatment with poly(N-isopropylacrylamide) significantly reduced the oxidative stress induced by free radicals (FIG. 9). This data indicates that the grafted solution has antioxidant properties.

### 6. Photoprotective Effect

To assess the photoprotective effect of poly(N-isopropylacrylamide) grafted onto hyaluronic acid on dermal cells, an MTT assay was performed using human epithelial cell line (HaCaT) after 24 hours of UVA irradiation. Results revealed that the solutions at concentrations of 0.5% and 5% improved cell survival rates compared to the group that received only UVA exposure (FIG. 10). These findings suggest that the grafted solution has photoprotective properties.

7. Enhancing Skin Permeability with Poly(N-isopropylacrylamide) Grafted onto Hyaluronic Acid

Poly(N-isopropylacrylamide) grafted onto hyaluronic acid can effectively deliver active ingredients deep into the dermis. In this study, a 3D simulated skin model (Episkin™) was utilized to assess the penetration abilities of fluorescein isothiocyanate (FITC), a derivative of fluorescein, as the active ingredient. The results demonstrated that poly(N-isopropylacrylamide) grafted onto hyaluronic acid significantly enhanced the penetration of FITC into the dermis of the Episkin™ model (see FIG. 11). These findings indicate that this grafted compound can promote the delivery of active ingredients into the dermal layer of the skin.

Effects of Poly(N-isopropylacrylamide) Grafted on Hyaluronic Acid in Combination with Other Agents

### 1. Enhanced Anti-Inflammatory Effect

1.1 Poly(N-isopropylacrylamide) Grafted on Hyaluronic Acid Shows 67% Anti-Inflammatory Effect When Used in Combination with Multi-X (Natural Active Ingredients) in Skin Cells

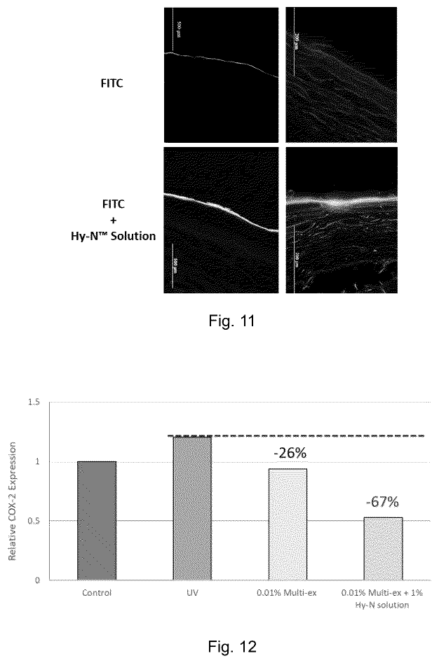

To investigate whether Poly(N-isopropylacrylamide) grafted on hyaluronic acid can enhance the anti-inflammatory effects of Multi-X in keratinocytes, the cells were incubated with a formulation of Poly(N-isopropylacrylamide) grafted on hyaluronic acid loaded with Multi-X for 24 hours prior to UV-A irradiation. The levels of COX-2 were then measured. The results indicated that the application of Multi-X at a concentration of 0.01% reduced COX-2 levels by approximately 26%. However, when Multi-X at 0.01% was combined with 1% Poly(N-isopropylacrylamide) grafted on hyaluronic acid, COX-2 expression was reduced by up to 67% compared to Multi-X alone (as shown in FIG. 12). These findings suggest that Poly(N-isopropylacrylamide) grafted on hyaluronic acid possesses synergistic anti-inflammatory properties when used alongside other active ingredients.

1.2 Poly(N-isopropylacrylamide) Grafted on Hyaluronic Acid Exhibits 85% Anti-Inflammatory Activity with Standardized Extracts

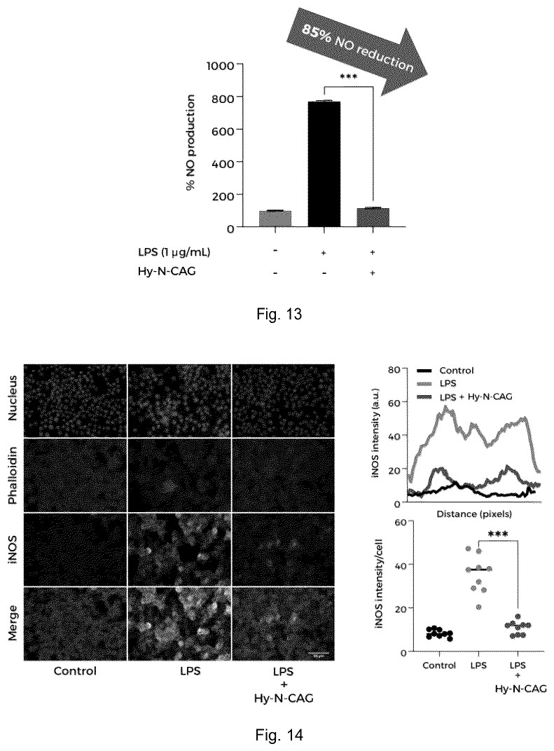

In another study, Poly(N-isopropylacrylamide) grafted on hyaluronic acid exhibited 85% anti-inflammatory activity when combined with standardized extracts of Centella asiatica, onion extract, and licorice extract (referred to as Hy-N-CAG) in white blood cells. The aim of this study was to evaluate the anti-inflammatory effects of this combination using the Griess assay. White blood cells were stimulated with Hy-N-CAG and lipopolysaccharide (LPS) at a concentration of 1 μg/mL for 24 hours. The generation of nitric oxide (NO) by cyclooxygenase (COX-2) and inducible nitric oxide synthase (iNOS) enzymes was measured using a Griess solution. The results revealed that Hy-N-CAG significantly reduced nitric oxide production by approximately 85% compared to LPS-stimulated cells, as illustrated in FIG. 13.

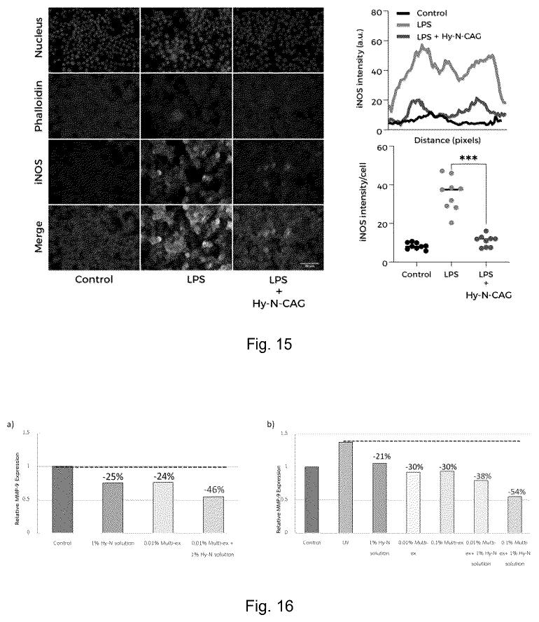

Additionally, the anti-inflammatory activity of Poly(N-isopropylacrylamide) grafted on hyaluronic acid loaded with the standardized extracts of Centella asiatica, onion extract, and licorice extract was further supported by immunofluorescence staining results. FIGS. 14 and 15 demonstrate the detection of iNOS and COX-2 enzymes, which appeared as green fluorescence in white blood cells. The analysis indicated that stimulation with LPS significantly increased the green fluorescence level compared to unstimulated cells. Poly(N-isopropylacrylamide) grafted on hyaluronic acid loaded with these extracts significantly reduced the expression of both iNOS and COX-2 enzymes in LPS-stimulated white blood cells. In conclusion, this combination demonstrates the potential to promote anti-inflammatory activity in white blood cells.

### 2. Enhanced Collagen Efficiency by Inhibiting Collagen Degradation

Poly(N-isopropylacrylamide) grafted on hyaluronic acid was shown to reduce MMP-9 expression by 46% when loaded with Multi-X (a natural active ingredient) in human epithelial cells and fibroblasts. To assess whether Poly(N-isopropylacrylamide) grafted on hyaluronic acid can enhance the anti-wrinkle efficacy of Multi-X in keratinocytes, the cells were incubated with this formulation for 24 hours prior to UV-A irradiation, after which MMP-9 levels were measured. The results indicated that a 0.01% concentration of Multi-X within a 1% Poly(N-isopropylacrylamide) grafted hyaluronic acid solution reduced MMP-9 expression by 46%, a greater reduction than observed with either the 1% hyaluronic acid-grafted Poly(N-isopropylacrylamide) solution or Multi-X alone (FIG. 16a). Additionally, in human fibroblast cells, 0.01% and 0.1% concentrations of Multi-X in the same solution reduced MMP-9 expression by approximately 38% and 54%, respectively (FIG. 16b). These data suggest that Poly(N-isopropylacrylamide) grafted on hyaluronic acid enhances the anti-wrinkle efficacy of other active ingredients.

### 3. Enhanced Antioxidant Activity

### 3.1 Poly(N-isopropylacrylamide) Grafted on Hyaluronic Acid Increases Antioxidant Activity of Cannabinoids by 1.2 Times

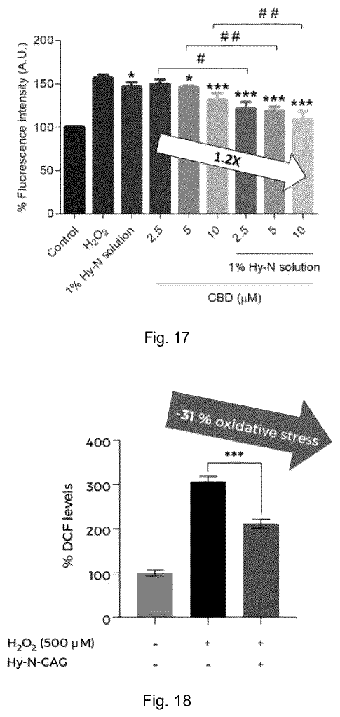

To investigate whether poly(N-isopropylacrylamide) grafted onto hyaluronic acid enhances the antioxidant activity of cannabinoids in keratinocytes, the cells were incubated with a solution of poly(N-isopropylacrylamide) grafted on hyaluronic acid loaded with cannabinoids for 24 hours. Following this, the cells were exposed to free radicals, and their oxidation status was measured post-treatment. The results indicated that the combination of poly(N-isopropylacrylamide) grafted on hyaluronic acid and cannabinoids effectively reduced oxidative stress compared to the untreated group. Specifically, using cannabinoids together with a 1% solution of poly(N-isopropylacrylamide) grafted onto hyaluronic acid significantly inhibited oxidative stress, showing an improvement of approximately 1.2 times compared to cannabinoids alone (see FIG. 17). These findings suggest that the poly(N-isopropylacrylamide) system can enhance the antioxidant activity of other active ingredients.

3.2 Poly(N-isopropylacrylamide) Grafted on Hyaluronic Acid Loaded with Centella Asiatica, Onion, and Licorice Extracts Improves Antioxidant Activity by 31%

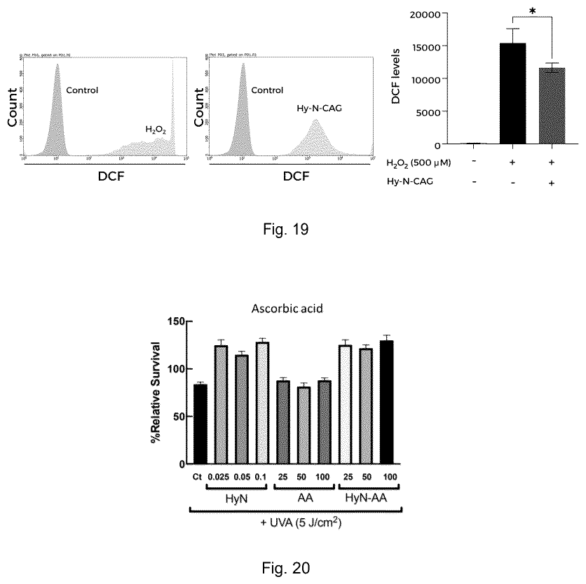

In a further study, poly(N-isopropylacrylamide) grafted onto hyaluronic acid and loaded with standardized extracts of Centella asiatica, onion, and licorice (referred to as Hy-N-CAG) demonstrated a 31% improvement in antioxidant activity compared to the extract without the grafted polymer. The DCFH-DA assay was employed to assess intracellular oxygen status in HaCaT keratinocyte cells, which were incubated with Hy-N-CAG for 24 hours before being exposed to 500 μM hydrogen peroxide to induce oxidative stress. Results showed that treatment with Hy-N-CAG significantly reduced DCF levels by 31% compared to cells treated with hydrogen peroxide alone. Flow cytometry confirmed that Hy-N-CAG reduced DCF levels in HaCaT cells. In conclusion, poly(N-isopropylacrylamide) grafted onto hyaluronic acid enhances protection against oxidative stress for various substances in keratinocytes, as illustrated in FIGS. 18 and 19.

### 4. Enhanced UVA Protection Efficiency

### 4.1 Ascorbic Acid Loaded in Poly(N-isopropylacrylamide) Grafted on Hyaluronic Acid Increases UVA Protection Efficiency by 1.5 Times

To explore whether poly(N-isopropylacrylamide) grafted onto hyaluronic acid can enhance UVA protection efficiency for ascorbic acid, keratinocytes were incubated with the poly(N-isopropylacrylamide) grafted on hyaluronic acid loaded with ascorbic acid (referred to as Hy-N™-AA) for 24 hours before UVA irradiation. The cell survival rate was then assessed through an MTT assay. The results indicated that ascorbic acid in the poly(N-isopropylacrylamide) formulation provided better protection against UVA radiation compared to ascorbic acid alone (see FIG. 20). These data suggest that poly(N-isopropylacrylamide) grafted onto hyaluronic acid enhances the sun protection efficacy of active ingredients.

### 4.2 Enhanced UVA Protection: Niacinamide

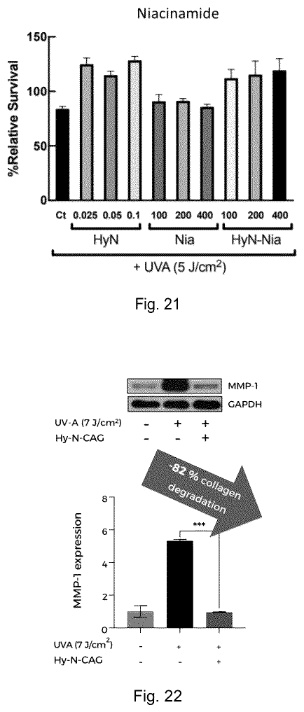

Further studies revealed that niacinamide loaded in poly(N-isopropylacrylamide) grafted onto hyaluronic acid also enhances UVA protection. Keratinocytes were incubated with ascorbic acid and niacinamide in the poly(N-isopropylacrylamide) formulation prior to UVA irradiation, and the cell survival rate was evaluated through an MTT assay. The results showed that niacinamide loaded in poly(N-isopropylacrylamide) grafted onto hyaluronic acid increased cell survival rates following UVA exposure compared to niacinamide alone (see FIG. 21). This data suggests that ascorbic acid in the poly(N-isopropylacrylamide) formulation can further enhance the sun protection efficacy of other active ingredients.

### 4.3 Enhanced UVA Protection: Standardized Centella Asiatica Extract, Onion Extract, and Licorice Extract (Hy-N-CAG)

This research examined the anti-aging effects of poly(N-isopropylacrylamide) grafted onto hyaluronic acid, which contains standardized extracts of Centella Asiatica, onion, and licorice (referred to as Hy-N-CAG). The study utilized keratinocytes and employed UV-A radiation as a stimulator of the aging process. Western Blot analysis was conducted to evaluate the expression of MMP-1, as illustrated in FIG. 22. The findings indicated that poly(N-isopropylacrylamide) grafted onto hyaluronic acid with standardized Centella Asiatica, onion, and licorice extracts (Hy-N-CAG) reduced the expression of the MMP-1 enzyme by 82%. This suggests that Hy-N-CAG possesses anti-aging properties in skin cells.

### 5. Enhanced Wound Healing

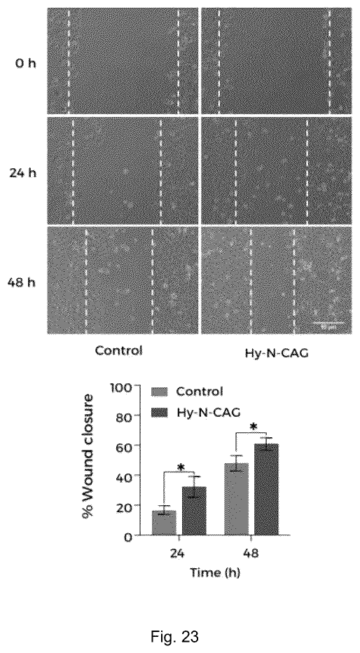

The study also investigated the wound healing properties of poly(N-isopropylacrylamide) grafted onto hyaluronic acid containing standardized extracts of Centella Asiatica, onion, and licorice (Hy-N-CAG) using an in vitro wound healing test technique. This technique is a standard method for studying the two-dimensional movement of cell groups. Monolayer keratinocyte cells were wounded using a 200 μL suction tip and then incubated with Hy-N-CAG for 48 hours. The wounds were subsequently recorded under a microscope. As shown in FIG. 23, poly(N-isopropylacrylamide) grafted onto hyaluronic acid with standardized extracts of Centella Asiatica, onion, and licorice (Hy-N-CAG) stimulated wound healing in keratinocytes at both 24 and 48 hours after wound formation.

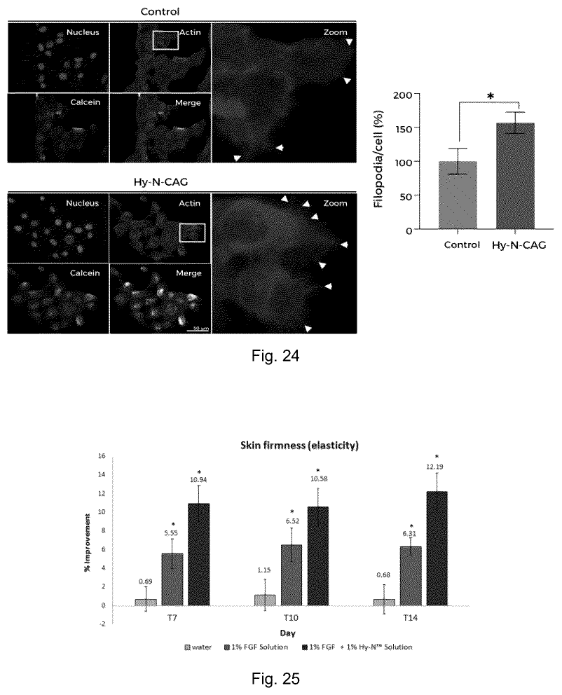

Additionally, further studies on cell morphology and filopodia formation were conducted. In these experiments, epidermal cell lines were pretreated with Hy-N-CAG for 24 hours and then stained with phalloidin-rhodamine. The results displayed in FIG. 24 demonstrate that Hy-N-CAG significantly increased the number of filopodia formed compared to untreated cells. These preliminary results suggest that poly(N-isopropylacrylamide) grafted onto hyaluronic acid with standardized extracts of Centella Asiatica, onion, and licorice (Hy-N-CAG) has the potential to enhance the efficacy of active ingredients in promoting wound healing in keratinocytes.

### Preliminary Clinical Results

### 1. Fibroblast Growth Factor (FGF)

This study involved nine healthy volunteers, with one participant eliminated on day 14. Samples were applied to the forearm twice daily for 14 days. The results indicated that 1% FGF combined with 1% poly(N-isopropylacrylamide) grafted onto 1% hyaluronic acid increased skin elasticity twofold compared to 1% FGF alone (FIG. 25). These clinical findings suggest that the addition of 1% poly(N-isopropylacrylamide) grafted onto hyaluronic acid may enhance the efficacy of FGF in cosmetic products.

### 2. Ascorbyl Glucoside (AA-2G, Stabilized Vitamin C)

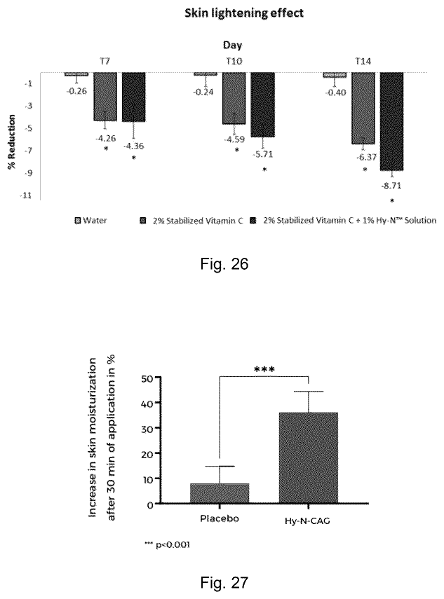

This study also included nine healthy volunteers, with one participant eliminated on day 14. The sample was applied to the forearm twice daily for 14 days. Results revealed that 2% stabilized vitamin C combined with 1% poly(N-isopropylacrylamide) grafted onto hyaluronic acid reduced the melanin index 1.3 times more effectively than 2% stabilized vitamin C alone (FIG. 26). These findings suggest that poly(N-isopropylacrylamide) grafted onto hyaluronic acid can enhance the anti-dark spot efficacy of vitamin C in skincare products.

3. Poly(N-isopropylacrylamide) Grafted on Hyaluronic Acid Loaded with Standardized Centella Asiatica Extract, Onion Extract, and Licorice Extract (Hy-N-CAG)

This study included seven volunteers (five females and two males) aged 22-33 years. The test product, Hy-N-CAG, was in gel form and compared with a placebo gel.

### 3.1 Skin Moisture

Experts measured skin moisture levels 30 minutes after application. Results showed a significant increase in skin moisture of 36.1% compared to baseline measurements, surpassing the placebo gel's increase of only 28.0% (FIG. 27). Hence, poly(N-isopropylacrylamide) grafted onto hyaluronic acid enhances the moisturizing effect on the skin.

### 3.2 Soothing Effect on Skin

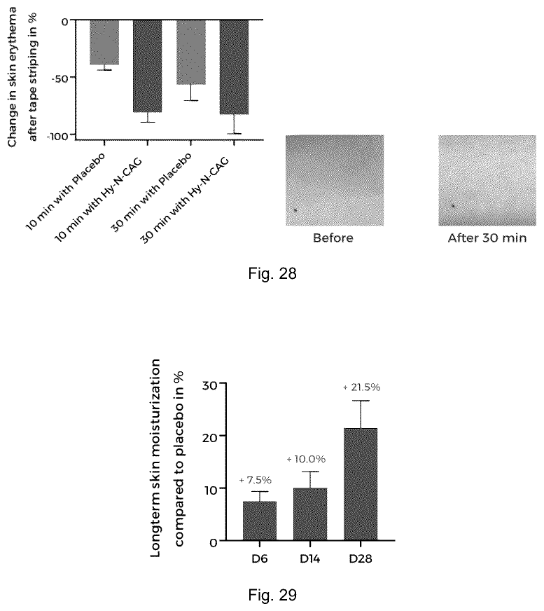

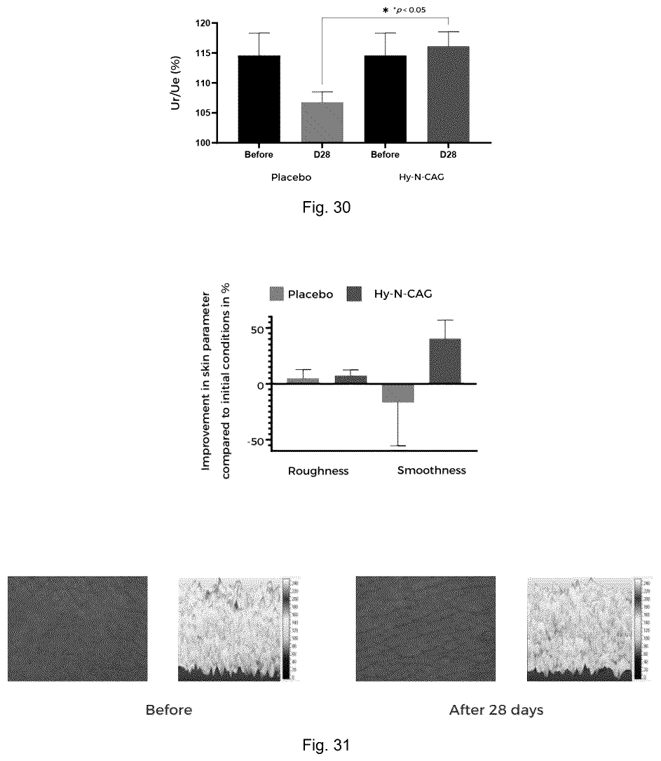

To evaluate the soothing effect, skin irritation was induced with tape, and the sample was applied to the irritated area. Redness levels were measured at 10 and 30 minutes post-application. The results showed a significant reduction in redness by 80% within 10 minutes and 82% within 30 minutes compared to the original skin condition (FIG. 28). This indicates that poly(N-isopropylacrylamide) grafted onto hyaluronic acid, supplemented with standardized Centella asiatica extract, onion extract, and licorice extract (Hy-N-CAG), effectively soothes irritated skin.

### 3.3 Long-Term Moisture Retention

Volunteers applied samples to their forearms morning and evening for 28 days. Skin moisture was measured on days 6, 14, and 28. The results indicated an increase in skin moisture of 7.5%, 10%, and 21.5% on days 6, 14, and 28, respectively, demonstrating that Hy-N-CAG improves long-term hydration.

### 3.4 Skin Elasticity

Experts evaluated skin elasticity before and after the 28-day application period. The results revealed that the poly(N-isopropylacrylamide) gel grafted onto hyaluronic acid loaded with standardized Centella asiatica extract, onion extract, and licorice extract (Hy-N-CAG) significantly improved skin elasticity compared to the placebo gel.

### 3.5 Skin Roughness

The roughness of the volunteers' skin was measured before and after 28 days of application. The results showed that the poly(N-isopropylacrylamide) gel grafted onto hyaluronic acid, containing standardized Centella asiatica extract, onion extract, and licorice extract (Hy-N-CAG), promoted skin smoothness by 7.6% and reduced roughness by 40.4% compared to baseline measurements.

### 3.6 Scar Roughness

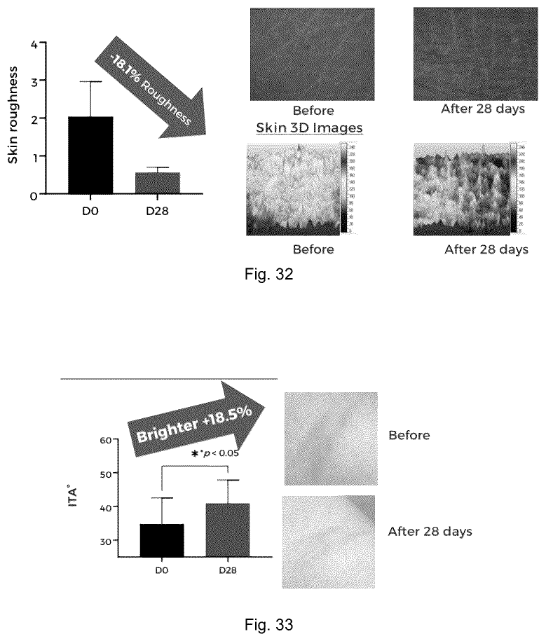

Experts measured the roughness of the scars before applying the treatment. The volunteers were then given samples of poly(N-isopropylacrylamide) gel, which was grafted onto hyaluronic acid and contained standardized extracts of Centella asiatica, onion, and licorice (Hy-N-CAG). This gel was applied to the test area twice a day for 28 days. At the end of this period, the experts measured the roughness of the scars again. The results indicated that the poly(N-isopropylacrylamide) grafted onto hyaluronic acid, along with the standardized extracts, reduced scar roughness by up to 18.1%, as shown in FIG. 32. Compared to the measurements taken before application, the results demonstrated that this treatment effectively promoted the smoothness of the scars.

### 3.7 Scar Darkening

The color of the scars was measured using a colorimeter, specifically by assessing the Individual Typology Angle (ITA) based on the L*a*b color space. Initially, experts measured the scar color before the treatment was applied. Afterward, volunteers applied the same poly(N-isopropylacrylamide) gel grafted onto hyaluronic acid with the standardized extracts twice a day for 28 days. Following this period, the color of the scars was measured again. The results, illustrated in FIG. 33, showed that the gel increased the brightness of the scars by 18.5% compared to measurements taken before treatment. This indicates the skin-brightening capability of poly(N-isopropylacrylamide) grafted onto hyaluronic acid.

4. Preliminary Clinical Trial of Poly(N-isopropylacrylamide) Grafted onto Hyaluronic Acid

This study involved 7 volunteers, consisting of 5 women and 2 men, aged 22 to 33 years. The test products included a serum containing 5% poly(N-isopropylacrylamide) grafted onto hyaluronic acid and a serum with 0.1% hyaluronic acid.

### 4.1 Skin Moisture

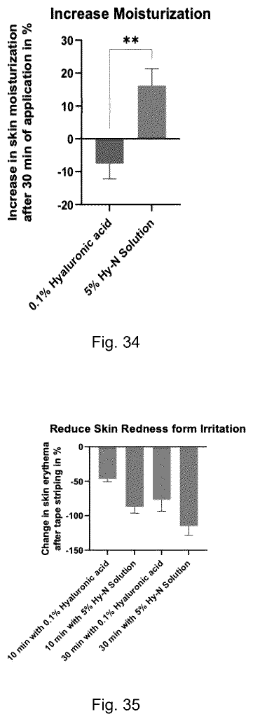

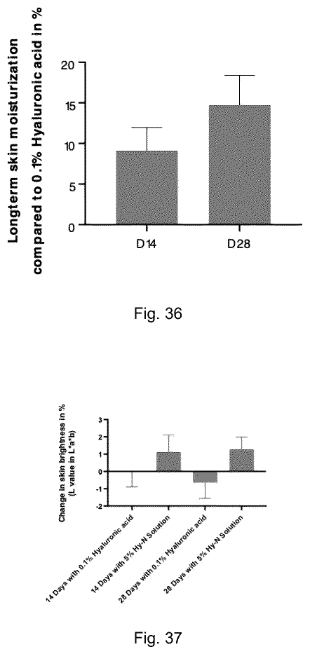

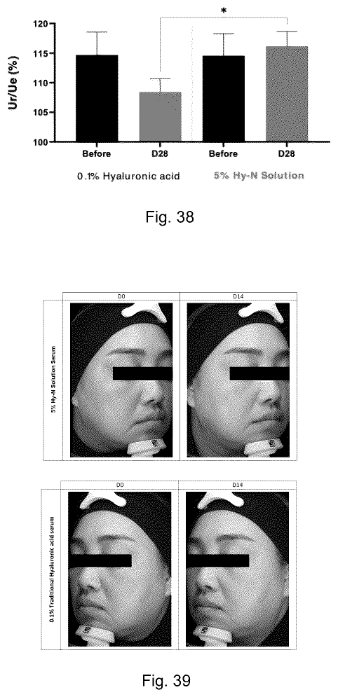

Experts measured skin moisture using a Corneometer before and after the volunteers applied the serum for 30 minutes, comparing the results with the group using the 0.1% hyaluronic acid serum. The findings revealed that skin moisture increased significantly by 16.2% after using the 5% poly(N-isopropylacrylamide) serum, while the group using 0.1% hyaluronic acid experienced a 23.8% increase (refer to FIG. 34). Thus, it can be concluded that the 5% poly(N-isopropylacrylamide) solution grafted onto hyaluronic acid provides rapid and effective hydration to the skin, outperforming traditional hyaluronic acid.

### 4.2 Soothing Effect

In this part of the study, skin irritation was induced using adhesive tape, followed by the application of the serum to the affected area. Experts evaluated the level of skin redness before and after the application at 10 and 30 minutes using a Mexameter. Results showed a significant reduction in redness of 87.3% within 10 minutes and 115.2% within 30 minutes, compared to the original condition before application (as shown in FIG. 35). Therefore, poly(N-isopropylacrylamide) grafted onto 5% hyaluronic acid effectively reduces redness and soothes irritated skin.

### 4.3 Long-Term Moisturizing Effect

Volunteers were instructed to apply the serum to their forearms in the morning and evening for 28 days. Skin moisture was measured using a Corneometer on days 14 and 28. Over this period, the results indicated an increase in skin moisture from before application by 9.15% on day 14 and 14.75% on day 28. This demonstrates that poly(N-isopropylacrylamide) grafted onto hyaluronic acid not only offers immediate hydration but also provides lasting moisture more effectively than standard hyaluronic acid.

### 4.4 Improvement of Skin Unevenness

Skin color was analyzed using a Colorimeter to record L*a*b values. Measurements were taken before the volunteers applied the serum to their forearms twice a day for 28 days. After this period, skin color was assessed again. The results (refer to FIG. 37) indicated that the 5% poly(N-isopropylacrylamide) grafted on hyaluronic acid improved skin brightness by 1.13% on day 14 and 1.29% on day 28, compared to the initial measurements, suggesting that this formulation enhances skin brightness.

### 4.5 Enhancement of Skin Elasticity

Skin elasticity was measured with a Cutometer before and after the 28-day application of the serum by the volunteers. The findings showed that poly(N-isopropylacrylamide) grafted onto 5% hyaluronic acid significantly improved skin elasticity compared to the 0.1% hyaluronic acid serum, highlighting the effectiveness of this formulation in enhancing skin elasticity.

5. Evaluation of the Efficacy of 5% Poly(N-Isopropylacrylamide) Grafted onto Hyaluronic Acid

A total of 22 volunteers (mean age 44±3 years, range 24-60 years) were randomly assigned to use two products: a 5% poly(N-isopropylacrylamide) grafted onto hyaluronic acid as the test product, and a 0.1% hyaluronic acid serum as the control.

### 5.1 Skin Moisture

Skin moisture was measured using a Corneometer® CM825 after 14 days of product use. The results indicated that the 5% poly(N-isopropylacrylamide) grafted onto hyaluronic acid increased skin moisture levels by an average of 10% (p<0.001), showing effectiveness in 82% of the volunteers.

### 5.2 Skin Clarity

The evaluation of skin brightness was conducted using a Skin Colorimeter CL 400, which measured L* (skin brightness) and b* (melanin content) parameters to assess changes in skin tone after 14 days of continuous product use. The results demonstrated that the 5% poly(N-isopropylacrylamide) grafted onto hyaluronic acid increased skin brightness (L*) by 1% (p=0.002) in 77% of volunteers, reduced melanin yellowness (b*) by 2% (p<0.001) in 95% of volunteers, and increased the ITA° by an average of +6% (p<0.001), observed in 77% of participants. In conclusion, the product displayed a significant effect on skin lightening, particularly in reducing melanin content.

### 5.3 Evaluation by VISIA© System

To gain clearer insights into the product's effects, the VISIA® System from CANFIELD® Imaging Systems was utilized. This technology analyzes skin conditions through multidimensional photography using different light sources, including white light, UV light, and polarized light. Volunteer sample number 05, as depicted in FIG. 39, exhibited the most noticeable results with the use of the 5% poly(N-isopropylacrylamide) grafted onto hyaluronic acid. The images captured by the VISIA® System demonstrated improvements in skin tone, smoothness, and moisture levels following product application. These experimental results align with the quantitative data from scientific measurements, further confirming that the 5% poly(N-isopropylacrylamide) grafted onto hyaluronic acid is highly effective in enhancing overall skin quality.

5.4 Feedback from Volunteers Who Participated in the Trial

All volunteers who took part in the trial provided feedback on the product's texture, smell, color, and their experience after use. Among the volunteers who used the 5% hyaluronic acid-grafted poly(N-isopropylacrylamide) serum, 100% reported overall satisfaction with the product. The color and smell were deemed good and satisfactory. The serum was quickly absorbed into the skin without leaving any stickiness.

After 14 days of use, every volunteer felt that the product effectively moisturized their skin, restored its vitality, and improved smoothness, tone, softness, and elasticity. Participants noted that their skin appeared radiant, clear, healthy, and less dull. The product also soothed the skin from external pollutants, making it feel comfortable. It was gentle and did not cause irritation. Furthermore, 100% of the volunteers expressed a desire to purchase the product regardless of its price and were willing to recommend it to friends and family.

6. The Effect of Product on Reducing Skin Redness from Chemical-Induced Inflammation

This study involved 22 volunteers (mean age 34±2 years, range 25-59 years) who were randomly assigned to use both products. The test product was the poly(N-isopropylacrylamide) serum grafted with 5% hyaluronic acid.

### 6.1 Skin Redness Measurement Using Spectrophotometer® CM700d**

After applying the products for 30 minutes, results showed that the poly(N-isopropylacrylamide) serum grafted with 5% hyaluronic acid reduced skin redness by an average of 64%. In comparison, the untreated area experienced a 70% reduction in redness. Although the results from both products were similar, there was no statistically significant difference between them.

Transepidermal water loss (TEWL) measurements taken using the Aquaflux™ instrument after 30 minutes of application indicated that the serum reduced TEWL by an average of 107%, while the untreated area recorded only a 9% reduction. Both products demonstrated high effectiveness in retaining skin moisture, but again, there was no statistical difference between them.

Skin temperature changes measured by an infrared camera after 30 minutes revealed that the serum reduced skin temperature by 95%, while the untreated area recorded a reduction of 104%. This also indicated no significant statistical difference between the two products.

The feedback from the volunteers reflected high satisfaction levels, with 100% finding the product's color appealing. Ninety-five percent noted that it absorbed quickly into the skin without leaving a sticky residue. Additionally, 55% liked the product's smell. Regarding effectiveness after 30 minutes of use, 95% of the volunteers felt it was comfortable on their skin, and 91% stated that it reduced skin irritation, helping restore a healthy appearance, particularly for those with dry skin.

Furthermore, 86% of the volunteers observed a reduction in redness, while 77% felt the product helped prevent water loss and 64% reported a significant increase in skin moisture. In conclusion, 95% of the volunteers indicated that they would purchase the product regardless of price and were willing to recommend it to others.

### 6.2 Infrared Thermography

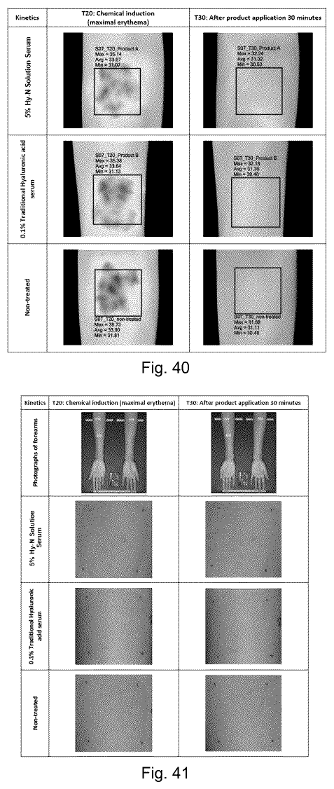

Infrared thermography is a valuable technique used to analyze surface temperature by measuring emitted heat. Thermal imaging cameras can assess temperature without making direct contact with the skin, allowing for real-time recording of results. This technology operates on the principle that objects emit infrared radiation, which can be converted into a thermal image displaying the skin's temperature distribution. In this study, we utilized the Fluke Ti480® Pro thermal imaging camera, known for its precise measurement of heat radiation with an error margin of less than 0.05° C. at a target temperature of 30° C. As illustrated in FIG. 40, using a thermal imaging camera enables effective monitoring of product outcomes, particularly in alleviating irritation and regulating skin temperature.

6.3 Macro Photography with a Digital Camera

This method captures detailed images of the skin's condition with high accuracy. Photography is conducted under controlled conditions regarding lighting, aperture, shutter speed, and camera distance to facilitate precise comparisons of results. To maintain consistency, the positions are meticulously monitored on the data processing screen, allowing for side-by-side comparison of images taken at different times. Macro photography effectively illustrates the impact of the product on skin condition, as shown in FIG. 41. It can be used to compare the effects of a poly(N-isopropylacrylamide) serum grafted onto 5% hyaluronic acid with those of a conventional 0.1% hyaluronic acid serum and untreated areas.

What is claimed is:

1. A method comprising: 1. Dissolution 2. Sonication and 3. Post-Preparation Storage
wherein the weight of Hy-N in the Dissolution process will be 0.10 grams, and the water will be 100 ml;
wherein a duration for sonication in the Sonication process will be approximately 15 to 30 minutes;
wherein the temperature in the Post-Preparation Storage process will be fixed at 4° C., and the duration for stabilizing the solution will be one night.

2. The method of claim 1, wherein the solution is stored after one night for stabilizing, will need to keep the temperature fixed at 4° C. all the time.

3. The method of claim 1, wherein the Hy-N solid before dilution in water must be kept at a temperature of −20° C. dry storage.

4. The method of claim 1, wherein the overnight mean is at least 12 hours after the Post-Preparation Storage process starts.
